(19) United States
(12) Patent Application Publication
(10) Pub. No.: US 2007/0021818 A1
(43) Pub. Date: Jan. 25, 2007

(54) Stent delivery device with anti-occluding positioning member

(76) Inventor: Joseph De Gregorio, Glen Rock, NJ (US)

Correspondence Address: MCCARTER & ENGLISH, LLP, FOUR GATEWAY CENTER, 100 MULBERRY STREET, NEWARK, NJ, 07102, US

(21) Appl. No.: 11/187,557

(22) Filed: Jul. 22, 2005

(51) Int. Cl. A61F 2/06

(52) U.S. Cl. 623/1.11

(57) ABSTRACT

A stent delivery device includes a distal balloon for supporting a stent in a deflated state and expanding the stent when inflated. A proximal balloon is located adjacent to the distal balloon. In a deflated configuration, the proximal balloon can pass through a diseased vessel. In an inflated configuration, the proximal balloon frictionally engages the diseased vessel while permitting at least some fluid flow through the diseased vessel and past the proximal balloon.

### FIELD OF THE INVENTION

The present invention relates to stent delivery systems, and, more particularly, to a stent delivery device having a proximal balloon for accurate positioning of a stent in the region of a stenosis.

### BACKGROUND OF THE INVENTION

With age, a large percentage of the population develops atherosclerotic arterial obstructions resulting in diminished blood circulation. The disturbance to blood flow that these obstructions cause may induce blood clots which further diminish or block the blood flow. When this process occurs in the coronary arteries it results in a heart attack. Presently, such obstructions are circumvented by surgically grafting a vessel to the blocked artery to bypass the obstruction (i.e., stenosis). Alternatively, arterial obstructions may be treated by angioplasty, a procedure by which a catheter equipped with an hydraulically expandable balloon is inserted through the arterial system over a flexible guidewire. Once the catheter has been properly positioned in the obstructed arterial lumen, the balloon is inflated to expand the obstructed lumen.

In some cases it is not enough to temporarily expand the obstructed lumen, since after removal of the balloon, the artery can become obstructed again in the same location. To keep the artery open relatively permanently, it is common to place an intravascular stent over the expandable balloon and deliver them to the obstructed lumen in their collapsed (i.e., non-expanded) states. After their expansion, the balloon is collapsed and withdrawn, while the stent remains in the artery in its expanded state to keep the artery open.

Self-expanding stents, which typically expand from a compressed delivery position to its original diameter when released from the delivery device, exert an outwardly-directed radial force on the constricted portion of the occluded arterial lumen. When the balloon is inflated, the self-expanding stent remains expanded to provide support for the arterial walls, keeping the arterial passage free for blood to flow therethrough. One common self-expanding stent is manufactured of Nitinol, a nickel-titanium shape memory alloy, which can be formed and annealed, deformed at a low temperature, and recalled to its original shape when heated, such as when deployed at body temperature in the body.

One problem that occurs with traditional stent delivery systems using balloon catheters is that it is difficult to place the stent in its proper position relative to the stenosis due to the beating of the heart, which causes the stent to move back and forth rapidly within the artery. U.S. patent application Ser. No. 10/293,002 by Squire et al. attempts to solve this problem by employing a dual balloon catheter system consisting of an outer catheter, which is equipped with an immobilizing balloon, and an inner catheter, which is equipped with a treatment balloon. An endovascular stent surrounds the treatment balloon, and the inner and outer catheters are coaxially-mounted relative to each other such that the inner catheter is movable relative to the outer catheter. In use, the outer catheter is introduced into a diseased vessel so as to position the immobilizing balloon near a treatment region (i.e., a vascular occlusion) of the diseased vessel. The immobilizing balloon has protrusions so that when it is inflated, the protrusions help to secure the immobilizing balloon to the diseased vessel and anchor the outer catheter in place. Treatment fluid is injectable between the immobilizing balloon and the treatment balloon (i.e., after the immobilizing balloon has been inflated and before expansion of the endovascular stent) through the end of the outer catheter. The position of the inner catheter relative to the outer catheter is then adjusted until the treatment balloon is properly positioned in the treatment region. The treatment balloon is then inflated, thereby expanding the endovascular stent against the vascular occlusion. The stent is left as a permanent scaffold to reinforce the vessel walls.

The dual balloon catheter system disclosed in the Squire et al. patent application has several limitations. For example, since the immobilizing balloon assumes a generally cylindrical shape when inflated, it blocks the flow of blood through the diseased vessel, and is therefore subject to being moved in the manner of a piston by the impingement of pressure arising from such blocked blood flow. In addition, the presence of such a flow-blocking device in the diseased vessel significantly inhibits injection of a radio-opaque contrast dye into the diseased vessel upstream of the immobilizing balloon to assist the surgeon in visualizing the position of the treatment balloon relative to the vascular occlusion. The present invention addresses these and other limitations of the prior art balloon-type catheter systems.

### SUMMARY OF THE INVENTION

The present invention overcomes many disadvantages and shortcomings of the prior art by providing a stent delivery device having a distal balloon and a proximal balloon positioned adjacent to the distal balloon. The distal balloon is adapted to support a stent in a deflated state and, upon inflation, to expand the stent. The proximal balloon, when deflated, has a configuration which permits its passage through a diseased vessel. Upon its inflation, the proximal balloon frictionally engages the diseased vessel without completely occluding or blocking fluid flow therethrough.

Further features and advantages of the invention will appear more clearly on a reading of the following detailed description of exemplary embodiments of the invention.

### BRIEF DESCRIPTION OF THE DRAWINGS

For a more complete understanding of the present invention, reference is made to the following detailed description of various exemplary embodiments thereof, considered in conjunction with the accompanying drawings, in which:

FIG. 1 is a schematic view of a system for treating stenoses in accordance with one exemplary embodiment of the present invention, including a catheter and a stent delivery device as they are being inserted into the vasculature of a patient;

FIG. 2 is a schematic view of the stent delivery device of FIG. 1 as it is being routed in a fully deflated state via a guide wire into the stenosed region of a coronary artery;

FIG. 3 is an enlarged scale view of FIG. 2, the coronary artery being shown in cross-section so as to illustrate the placement of the stent delivery device with respect to the stenosis;

FIG. 4 is a view similar to FIG. 3, except that a proximal balloon of the stent delivery device is shown after it has assumed an inflated state;

FIG. 5 is an end view of the stent delivery device of FIG. 2, and a sectional view of the coronary artery in which the device is positioned, as viewed according to section line 5--5 in FIG. 4, the stenosis S being shown in phantom to better illustrate the shape of the proximal balloon after its inflation;

FIG. 6 is a view similar to FIG. 4, except that the distal balloon of the stent delivery device is shown after it has assumed an inflated state, in which an associated stent is deployed (i.e., expanded) within the stenosis;

FIG. 7 is a view similar to FIG. 5, except that the stent delivery device depicted in FIG. 7 has been constructed in accordance with a second exemplary embodiment of the present invention; and

FIG. 8 is a view similar to FIG. 5, except that the stent delivery device depicted in FIG. 8 has been constructed in accordance with a third exemplary embodiment of the present invention.

### DETAILED DESCRIPTION OF THE INVENTION

For the purposes of the discussion below, “proximal” is defined as closer to or nearest the point of entry or the location of an operator of a treatment system including a stent delivery device. Conversely, “distal” is defined as further from the point of entry or the location of such an operator.

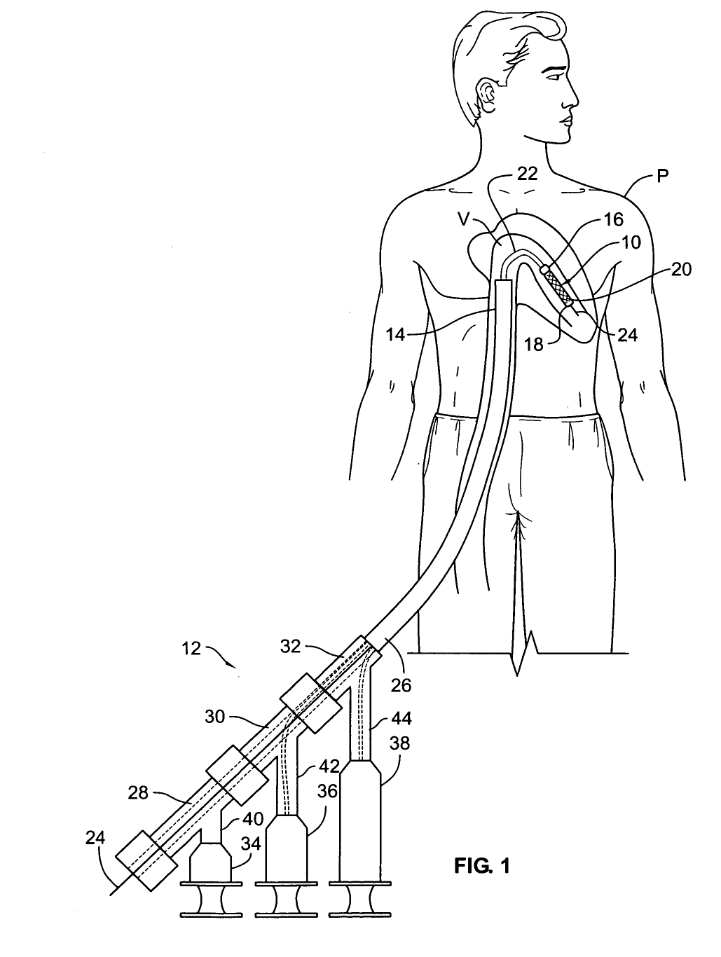

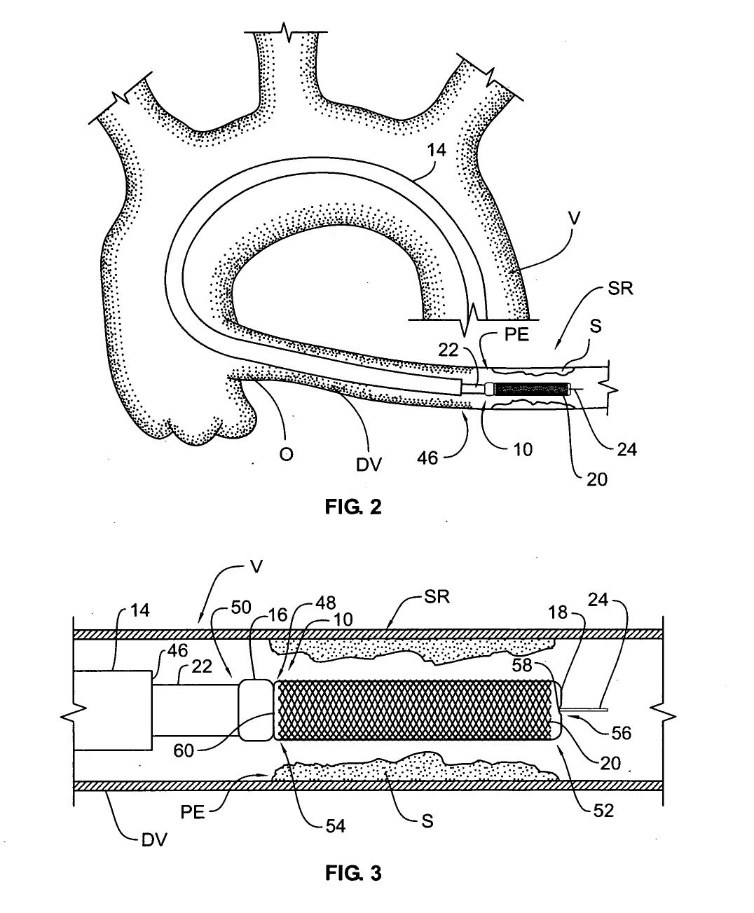

With reference to FIGS. 1 and 2, a stent delivery device 10 is utilized along with a treatment system 12 having a guiding catheter 14 which guides the stent delivery device 10 into the vasculature V of a patient P. The stent delivery device 10 has a proximal balloon 16, a distal balloon 18, and a stent 20 disposed around the distal balloon. The treatment system 12 further includes a flexible conduit 22 coupled to the stent delivery device 10 and contained for movement within the guiding catheter 14, and a guide wire 24 passing through the flexible conduit 22 and the stent delivery device 10, and along which the flexible conduit 22 and the stent delivery device 10 pass during their insertion into the vasculature V through the guiding catheter 14, which has a proximal end 26.

The treatment system 12 further includes ganged valve bodies 28, 30, 32, which are hermetically sealed for communication with the proximal end 26 of the guiding catheter 14, and respective syringes 34, 36, 38 associated with the valve bodies 28, 30, 32. The valve body 28 has a side port 40 through which a contrast fluid (e.g., a radio-opaque fluid) can be introduced into the guiding catheter 14 and injected therethrough into the vasculature V of the patient P by means of the syringe 34. The valve body 30, which is in communication with the distal balloon 18 of the stent delivery device 10 via a dedicated line (not separately shown) provided within the flexible conduit 22, has a side port 42 coupled to the syringe 36 for injecting or withdrawing a suitable fluid to inflate or deflate the distal balloon 18 of the stent delivery device 10. The valve body 32, which is in communication with the proximal balloon 16 of the stent delivery device 10 via a dedicated line (not separately shown) provided within the flexible conduit 22, has a side port 44 coupled to the syringe 38 for injecting or withdrawing a suitable fluid to inflate or deflate the proximal balloon 16 of the stent delivery device 10.

With reference to FIGS. 2 and 3, the stent delivery device 10 and the stent 20 are disposed at a distal end 46 of the guiding catheter 14 with the guide wire 24 extending therethrough. The guiding catheter 14 is inserted through the vasculature V into the ostium O of a diseased vessel DV (e.g., an obstructed coronary artery) by the Seldinger technique or other conventional methods known in the art. The guide wire 24 is advanced through the guiding catheter 14 and routed in a conventional manner through the diseased vessel DV such that the distal balloon 18 and the stent 20 are inserted into a stenosed region SR containing a stenosis S, while the proximal balloon 16 is moved into place at or near a proximal end PE of the stenosis S.

Referring in particular to FIG. 3. the proximal balloon 16 has a distal end 48 and a proximal end 50. Similarly, the distal balloon 18 has a distal end 52 and a proximal end 54. The guide wire 24 passes through the stent delivery device 10 via a passageway (not separately shown) extending continuously from the proximal end 50 of the proximal balloon 16 to the distal end 52 of the distal balloon 18 between an aperture or opening 56 that is surrounded by a lip seal 58 at the distal end 54 of the distal balloon 18 and another aperture or opening (not separately shown) at the proximal end 50 of the proximal balloon 16. The distal end 48 of the proximal balloon 16 shares a wall 60 with the proximal end 50 of the distal balloon 18, and the passageway (not separately shown) passes through the common wall 60. The wall 60 separates the proximal balloon 16 and the distal balloon 18 into two separate fluid-tight enclosures which may be inflated and/or deflated independently with respect to each other.

In preferred embodiments of the invention, the proximal balloon 16 and the distal balloon 18 integrally form a unitary structure. However, in alternative embodiments of the invention, the proximal balloon 16 and the distal balloon 18 are separate and distinct components, each one having a wall which abuts against the wall of the other, the abutting walls replacing the shaved or common wall 60. In some of these alternative embodiments, a medical professional can assemble the stent delivery device 10 on site by retrofitting a distal balloon with a proximal balloon.

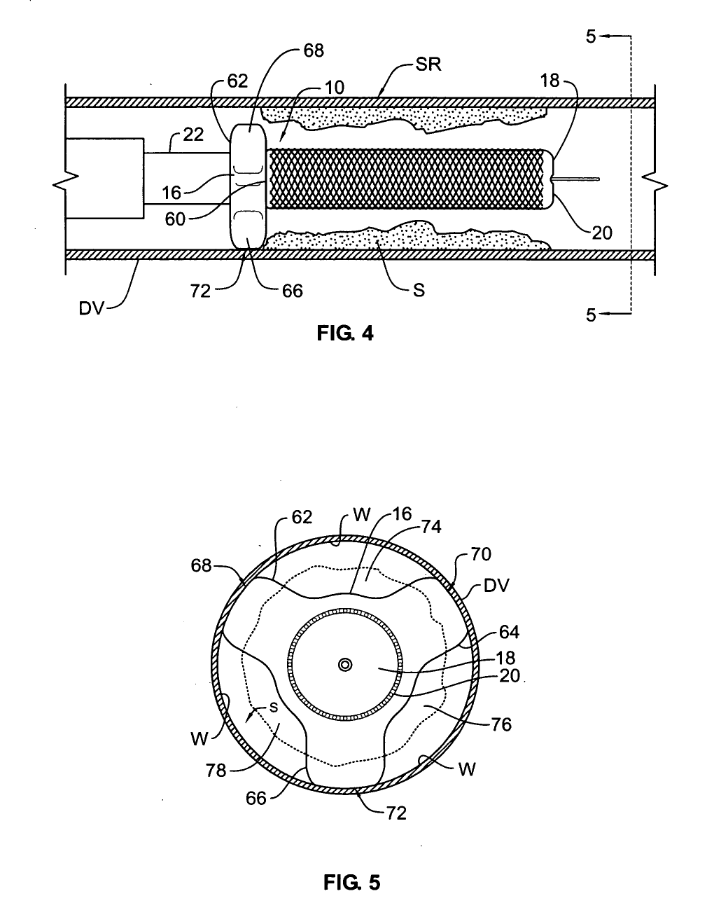

As will be discussed more fully hereinafter in connection with the operation of the stent delivery device 10, the proximal balloon 16, when inflated, assumes a lobed shape which includes lobes 62, 64, 66 (see FIGS. 4 and 5). The three lobes 62, 64, 66 of the inflated proximal balloon 16 extend radially outward beyond the periphery of the deflated distal balloon 18 and the unexpanded stent 20 and terminate in lobe ends 68, 70, 72, respectively, forming complementary passages in the form of gaps 74, 76, 78 between the lobes 62, 64, 66, such gaps 74, 76, 78 being circumferentially spaced, and sized and shaped so as to permit fluid flow past the inflated proximal balloon 16. The functions of the lobes 62, 64, 66, the lobe ends 68, 70, 72, and the gaps 74, 76, 78 will be explained more completely hereinafter.

With reference to FIG. 3, in operation, a contrast fluid is injected into the flow of blood (flowing from left to right in FIG. 3) within the diseased vessel DV starting at the distal end 46 of the catheter 14. The contrast fluid flows past the uninflated proximal and distal balloons 16, 18 and into and through the stenosed region SR, thereby aiding a practitioner in determining the position of the undeployed stent 20 relative to the stenosis S. Using real-time images of the stenosis S and the undeployed stent 20 provided via the contrast fluid, the practitioner, in preparing to treat the stenosis S with the stent 20, can execute positional corrections (e.g., using small, precisely controlled movements as necessary) until he or she has succeeded in placing the stent 20 in a desired position and/or alignment relative to the stenosis S. Similar and/or the same positional corrections can also be used to place the uninflated proximal balloon 16 in a desired position relative to (e.g., immediately adjacent to) the proximal end PE of the stenosed region SR.

Referring to FIGS. 4 and 5, once the stent 20 and/or the proximal balloon 16 of the stent delivery device 10 are properly positioned and/or aligned relative to the stenosed region SR as just described, the practitioner injects fluid into the flexible conduit 22 so as to inflate the proximal balloon 16, and thereby cause the proximal balloon 16 to become lodged in place within the diseased vessel DV upstream of the stenosis S. More particularly, fluid passing from the flexible conduit 22 into the proximal balloon 16 causes the lobes 62, 64, 66 of the proximal balloon 16 to inflate until the various circumferentially-spaced peripheral surfaces associated with the lobe ends 68, 70, 72 contact and frictionally engage the inside walls W of the diseased vessel DV, thereby effectively “anchoring” the proximal balloon 16 in place. Since the distal balloon 18 shares a wall 60 with the proximal balloon 16, its position within the diseased vessel DV is closely tied to that of the proximal balloon 16, such that the aforementioned “anchoring” of the proximal balloon 16 near the stenosis S indirectly, but quite effectively, locates the stent 20 in its desired position relative to the stenosis S.

If the position of the stent 20 still appears correct after inflation of the proximal balloon 16, the practitioner can decide to proceed with treatment of the stenosis S. However, if the position of the stent 20 now appears incorrect, the practitioner is free to reposition the stent 20 before expanding it. More particularly, the practitioner can, at any time during or after the inflation of the proximal balloon 16 but before the inflation of the distal balloon 18, continue to monitor the position of the stent 20 by continuing or restarting the flow of contrast fluid into the diseased vessel DV upstream of the proximal balloon 16. Referring again to FIG. 5, this is made possible, at least in part, because the collective size of the gaps 74, 76, 78 between the lobes 62, 64, 66 of the now-anchored proximal balloon 16 is large enough to permit at least some net blood flow (e.g., if not a continuous flow, then at least an intermittent flow) through the segment of the diseased vessel DV occupied by the now-inflated proximal balloon 16. Such blood flow allows the continued or restarted flow of contrast fluid to travel with it past the inflated proximal balloon 16 and into and through the stenosed region SR of the diseased vessel DV, thereby permitting continued beneficial imaging by the practitioner. If such continued imaging reveals the position of the stent 20 to be incorrect, the practitioner may choose to deflate the proximal balloon 16 and repeat one or more of the above-descibed stent positioning steps, as necessary, to produce the desired result.

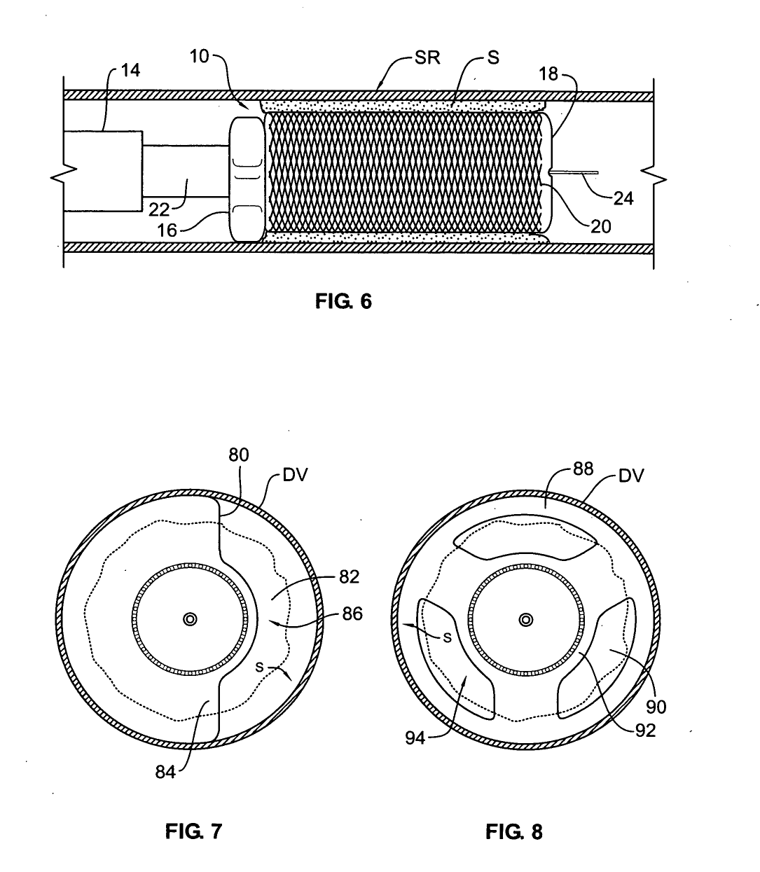

With reference to FIG. 6, fluid is now injected into the flexible conduit 22 to inflate the distal balloon 18 of the stent delivery device 10, thereby expanding the stent 20 within the stenosed region SR to a satisfactory degree relative to the stenosis S as is known in the art. Fluid is then withdrawn from the flexible conduit 22 so as to deflate the proximal and distal balloons 16, 18, while leaving the stent 20 in place in the stenosis S. The guide wire 24 is then withdrawn followed by the guiding catheter 14.

It should be understood that numerous advantages are provided by the stent delivery device 10 constructed in accordance with the foregoing description. For example, the passages in the form of gaps 74, 76, 78 between the lobes 62, 64, 66 in the proximal balloon 16 permit the flow of blood and contrast fluid even as the proximal balloon 16 serves to anchor the stent delivery device 10 in preparation for the placement of the stent 20. Furthermore, because the proximal and distal balloons 16, 18 share a common wall 60, the distal balloon 18 remains adjacent to the proximal balloon 16 at all times, reducing the possibility that flexure and/or stretching in the diseased vessel DV downstream of the anchoring point established by the proximal balloon 16 will result in an inaccurate placement of the stent 20. Still further, the practitioner is provided with the ability to positively confirm proper placement of the stent 16 after the proximal balloon 16 has been inflated, or conversely, to detect and correct for improper placement of the stent 20 before the placement of the stent 20, rather than being forced to accept a less-than-optimal stent position that becomes apparent only after the stent 20 has been irreversibly expanded.

It should also be noted that the stent delivery device 10 as discussed hereinabove in conjunction with FIGS. 1-6 can have many variations and modifications. For example, the number of lobes associated with the proximal balloon can be fewer than three. Alternatively, the number of lobes can exceed three in number so long as there are passages in the form of gaps between the lobes having a collective size to permit the flow of blood and contrast fluid. As shown in FIGS. 7 and 8, proximal balloons can be provided similar in all relevant respects to the proximal balloon 16 described above, and placed upstream of stenosis S of a diseased vessel DV in a similar manner to that described above, except that one such proximal balloon consists of a single lobe 80 forming a non-obstructing or non-occluding gap 82 (e.g., as with proximal balloon 84 of stent delivery device 86 shown in FIG. 7), and another such proximal balloon consists of a continuous peripheral lobe 88 provided with one or more passages or perforations 90 (e.g., as with proximal balloon 92 of stent delivery device 94 shown in FIG. 8). The proximal balloon 92 of FIG. 8, when in its inflated state, features interior walls which substantially entirely define or form the respective passages or perforations 90 for permitting fluid flow through the diseased vessel DV and past the proximal balloon 92, as well as a substantially continuous circumferential perimeter surface for frictionally engaging the inner walls of the diseased vessel DV. Further, in some embodiments of the present invention, the proximal balloon and distal balloon do not share a common wall, and in some such embodiments, the distal balloon, though not positioned immediately adjacent to the proximal balloon, is nevertheless positioned close enough thereto so as to facilitate accurate stent placement via the proximal balloon anchoring techniques described above. All such variations and modifications are intended to be included within the scope of the present invention as defined in the appended claims.

What is claimed is:

1. A stent delivery device, comprising a distal balloon for supporting a stent in a deflated state and for expanding the stent when inflated; and a proximal balloon positioned adjacent to said distal balloon, said proximal balloon having a deflated configuration, in which said proximal balloon can pass through a diseased vessel, and an inflated configuration, in which said proximal balloon frictionally engages the diseased vessel while permitting fluid flow through the diseased vessel and past said proximal balloon.

2. The stent delivery device of claim 1, wherein said proximal balloon, when in its said inflated configuration, is sized and shaped so as to at least partially define a passage for permitting fluid flow through the diseased vessel and past said proximal balloon.

3. The stent delivery device of claim 2, wherein said proximal balloon, when in its said inflated configuration, is sized and shaped such that said passage is formed by a gap between said proximal balloon and the diseased vessel.

4. The stent delivery device of claim 3, wherein said proximal balloon, when in its said inflated configuration, has at least one peripheral surface engageable with the diseased vessel so as to contribute to said frictional engagement between said proximal balloon and the diseased vessel.

5. The stent delivery device of claim 4, wherein said proximal balloon, when in its said inflated configuration, includes a plurality of peripheral surfaces arranged in a circumferentially spaced relationship to each other, each of said peripheral surfaces being engageable with the diseased vessel so as to contribute to said frictional engagement between said proximal balloon and the diseased vessel.

6. The stent delivery device of claim 5, wherein said proximal balloon, when in its said inflated configuration, includes a plurality of radially-extending lobes, each of said lobes including a corresponding one of said peripheral surfaces.

7. The stent delivery device of claim 6, wherein said proximal balloon, when in its said inflated configuration, is sized and shaped so as to define a plurality of passages arranged in a circumferentially spaced relationship to each other, each of said passages being adapted to permit fluid flow through the diseased vessel and past said proximal balloon.

8. The stent delivery device of claim 7, wherein said lobes are disposed between said passages so as to form an alternating circumferential arrangement of said lobes and said passages.

9. The stent delivery device of claim 2, wherein said proximal balloon, when in its said inflated configuration, is sized and shaped such that said passage is formed substantially entirely by interior walls of said proximal balloon.

10. The stent delivery device of claim 9, wherein said proximal balloon, when in its said inflated configuration, frictionally engages the diseased vessel along substantially an entire periphery of said proximal balloon.

11. The stent delivery device of claim 1, wherein said proximal balloon has a proximal end and a distal end opposite said proximal end, said distal balloon has a proximal end and a distal end opposite said proximal end, and said proximal balloon and said distal balloon share a common wall disposed forming said distal end of said proximal balloon and said proximal end of said distal balloon.

12. The stent delivery device of claim 11, further comprising a passageway passing through said common wall and extending from said proximal end of said proximal balloon to said distal end of said distal balloon, said passageway being sized and shaped so as to accept a guide wire for insertion along said passageway through said proximal and distal balloons.

13. The stent delivery device of claim 12, wherein said proximal balloon has an aperture at its said proximal end and said distal balloon has an aperture at its said distal end, said passageway extending continuously between said aperture of said proximal balloon and said aperture of said distal balloon.

14. The stent delivery device of claim 13, wherein said passageway is defined by internal walls of said proximal and distal balloons, respectively.

15. The stent delivery device of claim 14, wherein said proximal balloon, when in its said inflated configuration, is sized and shaped so as to at least partially define a passage for permitting fluid flow through the diseased vessel and past said proximal balloon.

16. The stent delivery device of claim 1, wherein said distal balloon and said proximal balloon are integrated to form a unitary structure.

17. The stent delivery device of claim 1, wherein said distal balloon and said proximal balloon are separate and distinct components which are in abutting relationship to one another.

18. The stent delivery device of claim 17, wherein said distal balloon is retrofitted with the proximal balloon.
